(19) United States
(12) Patent Application Publication
(10) Pub. No.: US 2010/0274203 A1
(43) Pub. Date: Oct. 28, 2010

(54) CONTAINER FOR TRANSDERMAL DELIVERY OF ACTIVE AGENT USING MICRONEEDLES

(75) Inventors: Seung Seob Lee, Daejeon (KR); Boo Joon Sul, Daejeon (KR); Man Hee Han, Daejeon (KR)

Correspondence Address: Edwards Angell Palmer & Dodge LLP, P.O. Box 55874, Boston, MA, 02205, US

(73) Assignee: MITI SYSTEMS INC., Daejeon (KR)

(21) Appl. No.: 12/826,009

(22) Filed: Jun. 29, 2010

Related U.S. Application Data
(63) Continuation of application No. PCT/KR2009/005741, filed on Oct. 8, 2009.

(30) Foreign Application Priority Data
Jan. 20, 2009 (KR) 10-2009-0004589

(51) Int. Cl. A61M 5/32

(52) U.S. Cl. 604/272

(57) ABSTRACT

A container for transdermal delivery of an active agent or agents using microneedles includes a reservoir adapted to receive a substance containing an active agent or agents; and a cover member adapted to cover an entrance of the reservoir, the cover member comprising one or more through-holes formed therein and one or more microneedles formed thereon. With the container, transdermal delivery can be made conveniently, easily, and effectively.

### CROSS-REFERENCE TO RELATED APPLICATION

This is a continuation of International Application PCT/KR2009/005741, with an international filing date of Oct. 8, 2009, which claims the benefit of Korean Application No. 10-2009-0004589 filed Jan. 20, 2009, the entire contents of which are incorporated herein by reference.

### TECHNICAL FIELD

The present invention relates to a container for use in transdermal delivery of an active agent.

### BACKGROUND ART

Methods of transdermally delivering an active agent (e.g., medical agent, cosmetic agent, nutritional agent, etc.) or agents to a subject were proposed. In some methods, an active agent or agents are applied on the skin of the subject. In some other methods, a pack, a patch, or the like containing an active agent or agents is attached to the skin. Such transdermal delivery methods, while being able to make the active agent bring into close contact with the skin simply and easily, does not allow the active agent to be sufficiently absorbed into the skin since the stratum corneum which is the outermost layer of the epidermis of the skin and is 10-60 μm in depth prohibits internal substances from being fluxed out and external substances from being fluxed in. Specifically, if the active agent is hydrophilic or has a high molecular weight, the transdermal absorption of the active ingredient further decreases.

As an alternative to the methods, methods of transdermal delivery by injection using a syringe needle having a diameter measured in millimeter units (mm) and a length measured in centimeter units (cm) were proposed. These methods have an advantage in that the syringe needle allows an active agent to be effectively delivered directly into the skin textures, but still involve drawback, for instance, in that the syringe needle stimulates a plurality of pain spots widely distributed in the skin, thereby resulting in a considerable pain to a subject in use and in that since performing the method requires a certain level of handling skills and experiences, thereby making it difficult for non-professionals to use.

In order to address and solve the above drawbacks, methods of transdermal delivery using a microneedle that has a diameter of several tens to a few hundreds of micrometers (μm) and a length of several tens to a few thousands of micrometers (μm) were proposed. Since the microneedle is relatively small in diameter and length as compared to the conventional syringe needles, the methods may reduce the pain that otherwise the subject would feel. Also, the methods may be used by not only professionals (e.g., nurses) but also non-professionals.

For example, U.S. Pat. No. 3,964,482 discloses a system for delivering an active agent, which includes a microneedle unit for puncturing the skin of a subject to increase transdermal delivery efficiency of the active agent and a reservoir integrally formed with the microneedle for containing the active agent. The system, however, requires a diaphragm or other means for preventing the active agent contained in the reservoir from leaking to the outside. Furthermore, the system requires a hollow microneedle and a microneedle having no channels but allowing for permeation of the drug that are hard to be mass-produced.

U.S. Pat. No. 6,537,242 teaches a microneedle unit coupled to a container such as, for example, a syringe. Such a system has an advantage in that it can deliver a certain quantity of an active agent, but still encounters a disadvantage in that its structure is complicated and its use consists of multiple steps, thus resulting in inconvenience of use. In addition, mass-production of the hollow microneedles required in the system is not easy to be mass-produced.

U.S. Patent Application Publication No. 2006/0051404 discloses a microstructure roller system including a plurality of hollow microneedles. Such a system has an advantage in that since a step of applying a roller and a step of applying an active agent are performed together, its use is easy and simple, but still entails a shortcoming in that mass-production of the hollow microneedles required in the system is not easy.

Korean Patent No. 753872 discloses a microneedle roller in which microneedles are formed on the outer circumferential surface of a roller head so that when the roller head is rolled on the skin, a plurality of micro-holes can be formed in the skin. Such a microneedle roller has an advantage in that its manufacture is relatively easy, but still entails a drawback in that a step of applying a roller and a step of applying an active agent are performed separately, thus resulting in inconvenience of use.

The above information disclosed in this Background section is only for enhancement of understanding of the background of the invention and therefore it may contain information that does not form the prior art that is already known in this country to a person of ordinary skill in the art.

### SUMMARY OF THE DISCLOSURE

The present invention has been made in order to solve the above-described problems occurring in the prior art, and it is an object of the present invention to provide a system for transdermal delivery of an active agent, in which a step of applying a microneedle and a step of delivering the active agent are performed together, thereby making its use easy and simple and simplifying manufacturing process.

In one embodiment, a container for transdermal delivery of an active agent or agents using microneedles comprises: a reservoir adapted to receive a substance containing an active agent or agents; and a cover member adapted to cover an entrance of the reservoir, the cover member comprising one or more through-holes formed therein and one or more microneedles formed thereon.

In another embodiment, the cover member may include: a cap adapted to be assembled and coupled to the entrance of the reservoir; and a microneedle substrate to be interposed between the cap and the top surface of the entrance of the reservoir. In this embodiment, the cap includes an opening formed centrally therein and the microneedle substrate includes one or more through-holes 22 formed therein and one or more microneedles 21 protrudingly formed thereon.

In still another embodiment, the container may further comprise an elastic layer interposed between the top surface of the entrance of the reservoir and the microneedle substrate. The elastic layer includes an opening formed centrally therein to correspond to the entrance of the reservoir.

The above and other features and advantages of the present invention will be apparent from or are set forth in more detail in the accompanying drawings, which are incorporated in and form a part of this specification, and the following Detailed Description, which together serve to explain by way of example the principles of the present invention.

### BRIEF DESCRIPTION OF THE DRAWINGS

FIGS. 1A and 1B illustrate an example of a container for transdermal delivery of an active agent or agents according to a first embodiment of the present invention;

FIGS. 2A and 2B illustrate an example of the cover member and the reservoir of the container of FIG. 1;

FIG. 3 is a perspective view illustrating an example of the cover member formed with guide grooves;

FIGS. 4A, 4B and 4C illustrate an example of a container for transdermal delivery of an active agent according to a second embodiment of the present invention; and

FIGS. 5A and 5B illustrate an example of a container for transdermal delivery of an active agent according to a third embodiment of the present invention.

Reference numerals set forth in the Drawings includes reference to the following elements as further discussed below:

| | |
|---|---|
| 10 reservoir | 11 entrance |
| 12 top surface | 13 recess |
| 20 cover member | 21 microneedle |
| 22 through-hole | 23 adhesive means |
| 24 cap | 25 opening |
| 26 microneedle substrate | 27 sealing means |
| 28 elastic layer | 29 guide grooves |

It should be understood that the appended drawings are not necessarily to scale, presenting a somewhat simplified representation of various preferred features illustrative of the basic principles of the invention. The specific design features of the present invention as disclosed herein, including, for example, specific dimensions, orientations, locations, and shapes will be determined in part by the particular intended application and use environment.

### DETAILED DESCRIPTION OF THE DISCLOSURE

Hereinafter, the present invention will be described in detail in connection with the preferred embodiments with reference to the accompanying drawings. However, these embodiments are for illustrative purposes, and the scope of the present invention is not limited thereto. Also, it will be understood by those skilled in the art that various modifications and variations can be made to the present invention without departing from the spirit and scope of the appended claims based on the illustrative embodiments.

As discussed above, the present invention provides a container for transdermal delivery of an active agent or agents. The container includes: a reservoir 10 adapted to receive a substance (e.g., drug, cosmetic substance, nutritional substance) containing an active agent or agents; and a cover member 20 adapted to cover the entrance 11 of the reservoir 10, the cover member 20 including one or more through-holes 22 formed therein and one or more microneedles 21 protrudingly formed thereon.

Any kind of active agent that can be delivered through the microneedle 21 may be used in the system. Examples of the active agent include medical agent, cosmetic agent, nutritional agent, and the like. The substance containing the active agent may be, for instance, a hormone formulation, a vitamin formulation and the like, and may be in the form of liquid or fine powder.

### Embodiment 1

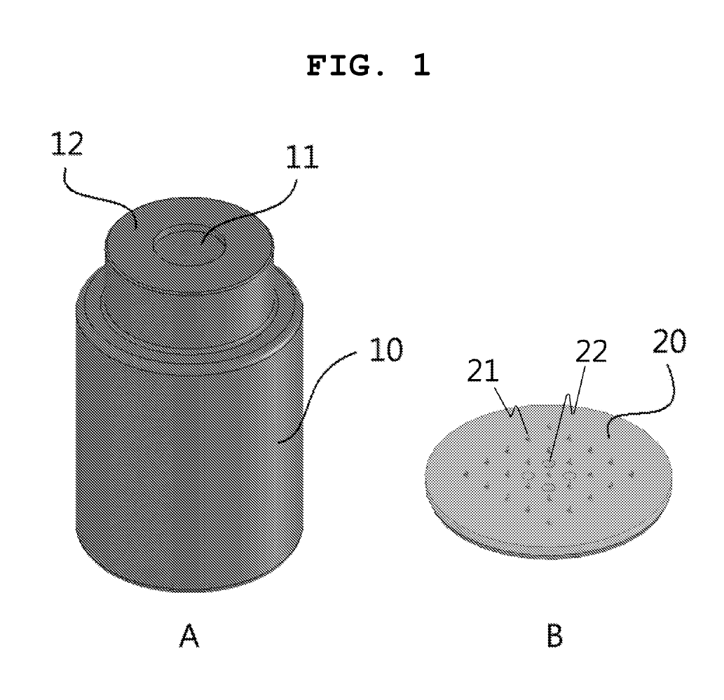

FIG. 1 is a perspective view illustrating an example of a container for transdermal delivery of an active agent or agents according to the first embodiment of the present invention.

FIG. 1A shows a reservoir 10 of the container. Although it is described in FIG. 1A that the reservoir 10 has a circular cross-section, the cross-sectional shape thereof is not limited thereto and may be suitably changed in view of convenience of use and/or aesthetical appearance and/or functional purpose, and so on.

FIG. 1B shows a cover member 20 of the container. The cover member 20 is coupled to the top surface 12 of the entrance 11 of the reservoir 10 and has a plurality of microneedles 21 formed on the top surface thereof so as to allow an active agent flowing out of the reservoir 10 to pass therethrough. The microneedle 21 can be manufactured by a method known in the art, and thus the detailed description of the structure thereof will be omitted for brevity.

Preferably, the cover member 20 includes at least one through-hole 22 formed therein through which a substance can flow out of the reservoir 10. If the number of the through-holes is too large, the substance excessively flows out of the reservoir, thereby decreasing transdermal drug delivery efficiency. Thus, the number of the through-holes should be properly adjusted and selected according to specific requirements.

Moreover, the area of the through-hole 22 preferably ranges between 1 mm² and 3 mm². If the area of the through-hole is smaller than 1 mm², a substance having a high viscosity may not flow out of the reservoir. On the contrary, if the area of the through-hole 22 is larger than 3 mm², a substance having a low viscosity may excessively flow out of the reservoir.

The size of the entrance 11 of the reservoir 10 may be set so as to cover the through-holes 22.

Suitably, the cover member 20 may be formed to be detachably coupled to the reservoir 10, in which case easy replacement thereof is ensured.

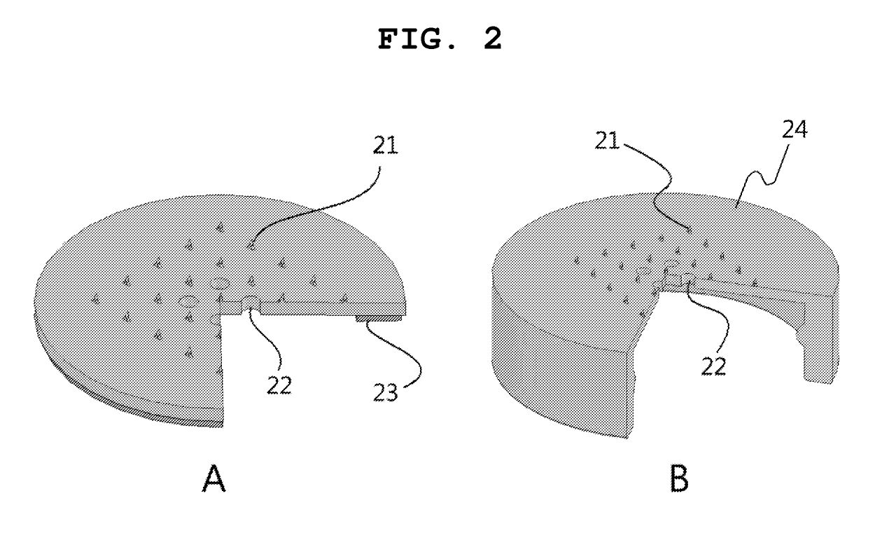

As an example, an adhesive means 23 such as, for example, an adhesive tape or the like may be formed on at least a portion of the bottom surface of the cover member as shown in FIG. 2A. As another example, the cover member 20 may be formed to have a cap 24 so that the cover member 20 can be assembled and coupled to the reservoir 10 as shown in FIG. 2B. As the assembling and coupling can be made by a method known in the art, detailed description thereof is omitted.

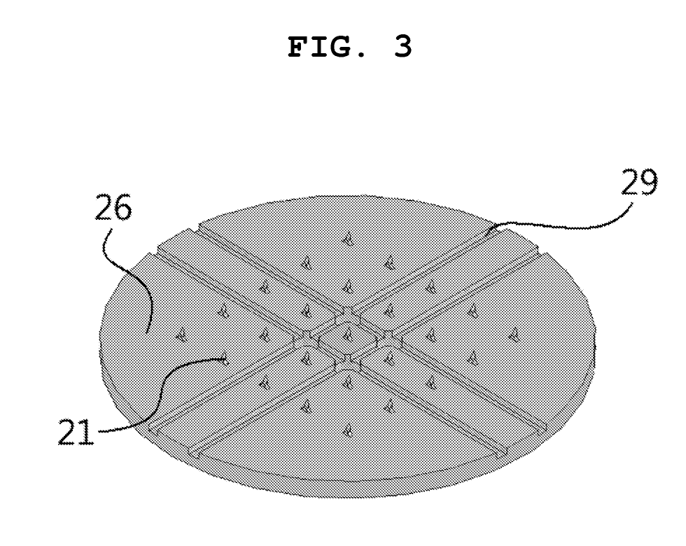

Also, preferably, the cover member 20 may further include guide grooves 29 formed on the top surface thereof so as to guide the flow of a substance containing an active agent as shown in FIG. 3. Since the active agent can be widely dispersed along the guide grooves 29, it can be conveniently and efficiently applied in an effective amount to a desired position of the subject. The guide grooves 29 may be also applied to the other embodiments as discussed below in a similar manner.

### Embodiment 2

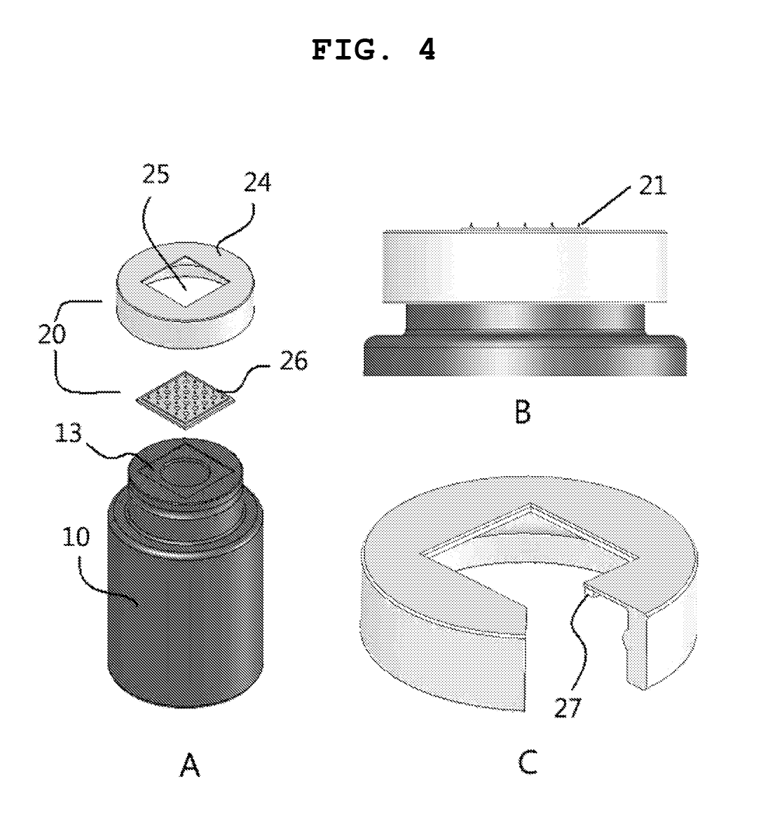

FIG. 4 is a perspective view illustrating an example of a container for transdermal delivery of an active agent or agents according to the second embodiment of the present invention.

The structure of the container according to the second embodiment is the basically similar to that of the container according to the first embodiment except for certain structural features as explained below. Detailed description of the same structure is thus omitted.

Referring to FIGS. 4A to 4C, in the second embodiment, the cover member 20 may include: a cap 24 adapted to be assembled and coupled to the entrance 11 of the reservoir 10, the cap including an opening 25 formed centrally therein; and a microneedle substrate 26 to be interposed between the cap 24 and the top surface 12 of the entrance of the reservoir 10, the microneedle substrate including one or more through-holes 22 formed therein and one or more microneedles 21 protrudingly formed thereon.

The microneedle substrate 26 can be made of any material if it is not harmful to the subject. The microneedle substrate 26 is preferably made of a polymer resin or a metal material.

The thickness of the microneedle substrate 26 may suitably be determined according to specific requirements. Preferably, it may range between 0.03 mm and 3 mm.

The microneedle substrate 26 may be manufactured to exhibit flexibility or inelasticity depending on the circumstances. While having a certain level of flexibility, the microneedle substrate 26 will need a sufficient strength such that it does not forcibly enter the inside of the reservoir 10 through the entrance 11 of the reservoir 10 with it rolled and twisted by being pushed by the skin while contacting the skin upon application of the container to the skin.

Preferably, the microneedle substrate 26 is formed so that when the reservoir, the microneedle substrate and the cap are assembled together, the top surface of the microneedle substrate 26 is placed at the same height as or higher than that of the cap 24.

In order for the active agent to effectively flow out of the reservoir through the opening 25 formed at the center of the cap 24, the opening 25 may be formed to have the same size as or larger than that of the entrance of the reservoir 10.

As shown in FIG. 4C, the cap may preferably further include a sealing means 27 disposed on at least a portion of the bottom surface thereof for preventing the active agent from unnecessarily flowing out of the reservoir through a portion other than the opening 25.

The reservoir 10 may include a recess 13 formed on the top surface 12 of the entrance thereof so as to allow the microneedle substrate 26 to be more easily mounted therein.

### Embodiment 3

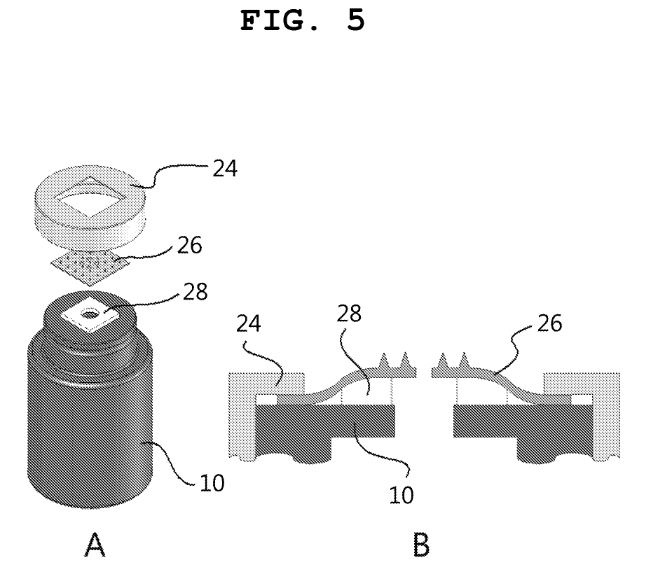

FIG. 5 is a perspective view illustrating an example of a container for transdermal delivery of an active agent or agents according to the third embodiment of the present invention.

The structure of the container according to the third embodiment is the basically similar to that of the container according to the second embodiment except for certain structural features as explained below. Detailed description of the same structure is thus omitted.

Referring to FIG. 5A, a container according to the third embodiment further includes an elastic layer 28 formed on the top surface 12 of the entrance 11 of the reservoir 10.

FIG. 5B shows a vertical cross-sectional view of the cover member 20 assembled and coupled to the reservoir.

The central portion of the microneedle substrate 26 is bent upwardly in a convex shape by means of the elastic layer 28, so that since the microneedles are protruded higher than the top surface of the cap 24, the contact between the container and the skin can be more easily made. Besides, the elastic layer 28 interposed between the top surface of the entrance of the reservoir 10 and the microneedle substrate 26 also serves to prevent leakage of the active agent between the microneedle substrate 26 and the reservoir 10.

Preferably, the container according to at least one of the embodiments may further include one or more additional caps for covering the cover member 20 so as to prevent contamination of the microneedles 21 while being kept in custody and unnecessary leakage of the active ingredient.

According to the present invention, it is possible to provide a container for transdermal delivery of an active agent or agents that can perforate the stratum corneum layer of the skin using microneedles and deliver the active agent or agents therethrough efficiently, conveniently and easily.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

1. A container for transdermal delivery of an active agent or agents using microneedles, the container comprising:
a reservoir adapted to receive a substance containing an active agent or agents; and
a cover member adapted to cover an entrance of the reservoir, the cover member comprising one or more through-holes formed therein and one or more microneedles formed thereon.

2. The container according to claim 1, wherein the total area of the through-holes is less than 3 mm².

3. The container according to claim 1, wherein the cover member includes a guide groove formed on the top surface thereof so as to guide the flow of the substance containing the active agent or agents.

4. The container according to claim 1, wherein the cover member is detachably coupled to the top surface of the entrance of the reservoir.

5. The container according to claim 4, wherein the total area of the through-holes is less than 3 mm².

6. The container according to claim 4, wherein the cover member includes a guide groove formed on the top surface thereof so as to guide the flow of the substance containing the active agent or agents.

7. The container according to claim 1, wherein the cover member comprise:
a cap assembled and coupled to the entrance of the reservoir, the cap including an opening formed centrally therein; and
a microneedle substrate interposed between the cap and the top surface of the entrance of the reservoir, the microneedle substrate including one or more through-holes formed therein and one or more microneedles protrudingly formed thereon.

8. The container according to claim 7, wherein the total area of the through-holes is less than 3 mm².

9. The container according to claim 7, wherein the cover member comprises guide grooves formed on the top surface thereof so as to guide the flow of the substance containing the active agent or agents.

10. The container according to claim 7, further comprising an elastic layer interposed between the top surface of the entrance of the reservoir and the microneedle substrate, the elastic layer including an opening formed centrally therein to correspond to the entrance of the reservoir.

11. The container according to claim 10, wherein the total area of the through-holes is less than 3 mm².

12. The container according to claim 11, wherein the cover member comprises guide grooves formed on the top surface thereof so as to guide the flow of the substance containing the active agent or agents.

13. The container according to claim 1, further comprising at least one additional cap for covering the cover member.
